SEQUENCE LISTING (22 sequences)

SEQ ID NO: 1 - DNA, 1369 bp, Hafnia alvei  ATCC 51873
tgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttggtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgcaacgagcgcaacccttatcctttgttgccagcacgtcatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttacctaaaga

SEQ ID NO: 2 - DNA, 1488 bp, Hafnia alvei ATCC 13337
  misc_feature (339)..(339): n is a, c, g, or t
  misc_feature (930)..(930): n is a, c, g, or t
  misc_feature (965)..(965): n is a, c, g, or t
  misc_feature (1480)..(1480): n is a, c, g, or t
  misc_feature (1482)..(1482): n is a, c, g, or t
  misc_feature (1484)..(1485): n is a, c, g, or t
attgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgaygtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactnctacgggaggcagcagtggggaatattgcacaatgggcgcaagcctgatgcagccatgccgcgtgtatgaagaaggccttcgggttgtaaagtactttcagcgaggaggaaggcattgtggttaataaccacagtgattgacgttactcgcagaagaagcaccggctaactccgtgccagcagccgcggtaatacggagggtgcaagcgttaatcggaattactgggcgtaaagcgcacgcaggcggttgrttaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggccngcacaagcggtggagcatgtggtttaattcgatgnaacgcgaagaaccttacctactcttgacatccagagaatttagcagagatgctttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcangncnnggg

SEQ ID NO: 3 - DNA, 1543 bp, Hafnia alvei FB1
taattgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgatgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattgtggttaataaccgcagtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttagcagagatgctttagtgccttcgggaactctgagacaggtgctgcatggctgtcgtcagctcgtgttgtgaaatgttgggttaagtcccgcaacgagcgcaacccttatcctttgttgccagcgcgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 4 - DNA, 1543 bp, Hafnia alvei strain NCTC6578
taattgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgacgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattgtggttaataaccacagtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttagcagagatgctttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 5 - DNA, 1132 bp, Hafnia alvei PCM 1202
taattgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgacgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattgtggttaataaccgcagtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttagcagagatgctttagtgccttcgggaactctgagacaggtgctgcatggctgtcgtcagctcgtgttgtgaaatgttgggttaagtcccgcaacgagcgcaacccttatcctttgttgccagc

SEQ ID NO: 6 - DNA, 1543 bp, Hafnia alvei strain A23BA
taattgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgacgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattgtggttaataaccacagtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgcaacgagcgcaacccttatcctttgttgccagcgcgtaatggcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 7 - DNA, 1553 bp, Hafnia alvei strain HUMV-5920
tttaattgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgacgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattgtggttaataaccgcagtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgcaacgagcgcaacccttatcctttgttgccagcgcgtaatggcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttacctaaaga

SEQ ID NO: 8 - DNA, 1543 bp, Hafnia alvei strain FDAARGOS_1038
taattgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggaggggggtaactactggaaacggtagctaataccgcatgacgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttagtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgcaacgagcgcaacccttatcctttgttgccagcacgtcatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 9 - DNA, 1543 bp, Hafnia paralvei ATCC 29927
taattgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgacgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttagtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 10 - DNA, 1543 bp, Hafnia paralvei strain AVS0177
taattgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgacgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 11 - DNA, 1074 bp, Hafnia sp. HMSC23F03
ccttagtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgctagagatagcttagtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 12 - DNA, 1542 bp, Escherichia coli CFT073
aaattgaagagtttgatcatggctcagattgaacgctggcggcaggcctaacacatgcaagtcgaacggtaacagaaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 13 - DNA, 1508 bp, Escherichia coli Nissle 1917
aaattgaagagtttgatcatggctcagattgaacgctggcggcaggcctaacacatgcaagtcgaacggtaacaggaatcagcttgctgatttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagagtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgatctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 14 - DNA, 1543 bp, Escherichia coli O2H1
aaattgaagagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccaggggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 15 - DNA, 1543 bp, Escherichia coli FVEC1302
aaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaacttttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 16 - DNA, 1552 bp, Escherichia sp. E4742
ttctttaaggtaaggaggtgatccaaccgcaggttcccctacggttaccttgttacgacttcaccccagtcatgaatcacaaagtggtaagcgccctcccgaaggttaagctacctacttcttttgcaacccactcccatggtgtgacgggcggtgtgtacaaggcccgggaacgtattcaccgtggcattctgatccacgattactagcgattccgacttcatggagtcgagttgcagactccaatccggactacgacgcactttatgaggtccgcttgctctcgcgaggtcgcttctctttgtatgcgccattgtagcacgtgtgtagccctggtcgtaagggccatgatgacttgacgtcatccccaccttcctccagtttatcactggcagtctcctttgagttcccggccggaccgctggcaacaaaggataagggttgcgctcgttgcgggacttaacccaacatttcacaacacgagctgacgacagccatgcagcacctgtctcacggttcccgaaggcacattctcatctctgaaaacttccgtggatgtcaagaccaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacggcgtggactaccagggtatctaatcctgtttgctccccacgctttcgcacctgagcgtcagtcttcgtccagggggccgccttcgccaccggtattcctccagatctctacgcatttcaccgctacacctggaattctacccccctctacgagactcaagcttgccagtatcagatgcagttcccaggttgagcccggggatttcacatctgacttaacaaaccgcctgcgtgcgctttacgcccagtaattccgattaacgcttgcaccctccgtattaccgcggctgctggcacggagttagccggtgcttcttctgcgggtaacgtcaatgagcaaaggtattaactttactcccttcctccccgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccgttaccccacctactagctaatcccatctgggcacatccgatggcaagaggcccgaaggtccccctctttggtcttgcgacgttatgcggtattagctaccgtttccagtagttatccccctccatcgggcagtttcccagacattactcacccgtccgccactcgtcagcgaatcagcaagctgattcctgttaccgttcgacttgcatgtgttaggcctgccgccagcgttcaatctgagccatgatcaaactcttcaattt

SEQ ID NO: 17 - DNA, 1554 bp, Escherichia sp. E4694
aaattgaagagtttgatcatggctcagattgaacgctggcggcaggcctaacacatgcaagtcgaacggtaacaggaatcagcttgctgattcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttaccttaaagaagc

SEQ ID NO: 18 - DNA, 1523 bp, Escherichia sp. E4702
agagtttgatcatggctcagattgaacgctggcggcaggcctaacacatgcaagtcgaacggtaacaggaaacagcttgctgtttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggcctcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttgga

SEQ ID NO: 19 - DNA, 1543 bp, Obesumbacterium proteus ATCC 12841
taattgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcatgatgtcttcggaccaaagtgggggaccttcgggcctcacgccatcagatgtgcccagatgggattagctagtaggtggggtaatggctcacctaggcgacgatctctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattgtggttaataaccgcagtgattgacgttactcgcagaagaagcaccggctaactccgtgccagcagccgcggtaatacggagggtgcaagcgttaatcggaattactgggcgtaaagcgcacgcaggcggttgattaagtcagatgtgaaatccccgagcttaacttgggaactgcatttgaaactggtcagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttagcagagatgctttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 20 - DNA, 1542 bp, Obesumbacterium proteus PCM 1204
taaggaggtgatccaaccgcaggttcccctacggttaccttgttacgacttcaccccagtcatgaatcacaaagtggtaagcgccctcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacatactttatgaggtccgcttgctctcgcgagttcgcttctctttgtatatgccattgtagcacgtgtgtagccctactcgtaagggccatgatgacttgacgtcatccccaccttcctccggtttatcaccggcagtctcctttgagttcccaccattacgtgctggcaacaaaggataaggttgcgctcgttgcgggacttaacccaacatttcacaacacgagctgacgacagccatgcagcacctgtctcagagttcccgaaggcactaaagcatctctgctaaattctctggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacagcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactctagctgaccagtttcaaatgcagttcccaagttaagctcggggatttcacatctgacttaatcaaccgcctgcgtgcgctttacgcccagtaattccgattaacgcttgcaccctccgtattaccgcggctgctggcacggagttagccggtgcttcttctgcgagtaacgtcaatcactgcggttattaaccacaatgccttcctcctcgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagagatcgtcgcctaggtgagccattaccccacctactagctaatcccatctgggcacatctgatggcgtgaggcccgaaggtcccccactttggtccgaagacgtcatgcggtattagctaccgtttccagtagttatccccctccatcaggcagtttcccagacattactcacccgtccgccgctcgtcacccagagagcaagctctcttgtgctaccgctcgacttgcatgtgttaggcctgccgccagcgttcaatctgagccatgatcaaactcttcaatta

SEQ ID NO: 21 - DNA, 1542 bp, Sodalis sp. dw_96
taattgaagagtttgatcatggctcagattgaacgctggcggcaggcctaacacatgcaagtcgagcggcagcggaaagtagcttgctactttgccggcgagcggcggacgggtgagtaatgtctggggatctgcccgatggagggggataactactggaaacggtagctaataccgcataacgtcgcaagaccaaagtgggggaccttagggcctcacaccatcggatgaacccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaagggttgggttgaatagattcaatcattgacgttacccgcagaagaagcaccggctaactccgtgccagcagccgcggtaatacggagggtgcaagcgttaatcggaattactgggcgtaaagcgtgcgtaggcggttcgttaagtctgttgtgaaagccccgggctcaacctgggaatggcaatggatactggcgagctagagtgtgtcagaggatggtggaattcctggtgtagcggtgaaatgcgtagatatcaggaggaacaccagtggcgaaggcggctctctgggccgtaactgacgctgaggcacgaaagcgtggggagcaaacaggattagaaaccccagtagtccatgctgtaaacgatgtcgatttgaaggttgtggccttgagccgtggctttcggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccacaggaatttgcagagatgcgaatgtgccttcgggaactgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgacaaaccggaggaaggtggggatgacgtcaagtcatcatggcccttacgagtagggctacacacgtgctacaatggcgcatacaaagagaagcgaactcgcgagagccagcggacctcacaaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 22 - DNA, 1541 bp, Sodalis sp. dw_23
taattgaagagtttgatcatggctcagattgaacgctggcggcaggcctaacacatgcaagtcgagcggcagcgggaagtagcttgctactttgccggcgagcggcggacgggtgagtaatgtctggggatctgcccgatggagggggataactactggaaacggtagctaataccgcataacgtcgcaagaccaaagtgggggaccttagggcctcacaccatcggatgaacccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggggttgggttgaatagattcaatcattgacgttacccgcagaagaagcaccggctaactccgtgccagcagccgcggtaatacgtagggtgcaagcgttaatcggaattactgggcgtaaagcgtgcgcaggcggttttgtaagacagtgtgaaatccccgggctcaacctgggaactgcattgtgactgcaaggctagagtacggcagaggggggtggaattccacgtgtagcagtgaaatgcgtagatatgcggaggaacaccgatggcgaaggcagccccctgggtctgtactgacgctcatgcacgaaagcgtggggagcaaacaggattagataccctggtagtccacgctgtaaacgatgtcgatttgaaggttgtggccttgagccgtggctttcggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaagacttcagagatggagttgtgccttcgggagctctgagacaggtgctgcatggctgtcgtcagctcgtgttgtgaaatgttgggttaagtcccgcaacgagcgcaacccttatcctttgttgccagcacgtaatggtgggaactcaaaggagactgccggtgacaaaccggaggaaggtggggatgacgtcaagtcatcatggcccttacgagtagggctacacacgtgctacaatggcgcatacaaagagaagcgaactcgcgagagccagcggacctcacaaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

(19) United States
(12) Patent Application Publication
(10) Pub. No.: US 2024/0226189 A1
(43) Pub. Date: Jul. 11, 2024

(54) MICROBIOME MICROORGANISM AND USES THEREOF

(71) Applicant: BIOMICA LTD., Rehovot (IL)

(72) Inventors: Osnat TIROSH, Ness Ziona (IL); Sheerli KRUGER BEN SHABAT, Raanana (IL); Shiri MESHNER, Raanana (IL); Elran HABER, Kiryat Ono (IL); Shiri ESHAR, Hulda (IL); Evyatar BRAYER, Tel Aviv (IL); Yehuda RINGEL, Tel Aviv (IL)

(73) Assignee: BIOMICA LTD., Rehovot (IL)

(21) Appl. No.: 18/290,240

(22) PCT Filed: May 12, 2022
(86) PCT No.: PCT/IL2022/050504

§ 371 Date: Nov. 10, 2023

(30) Foreign Application Priority Data
May 12, 2021 (IL) 283124

(51) Int. Cl. A61K 35/74; C12N 1/20; C12R 1/01

(52) U.S. Cl. CPC A61K 35/74; C12N 1/205; C12R 2001/01

(57) ABSTRACT

The present invention provides a microbiome microorganisms, microbial consortium, compositions and kits comprising the same and uses thereof.

### TECHNOLOGICAL FIELD

The present discourse relates to microbiome microorganism, compositions and kits comprising the same and uses thereof.

### BACKGROUND ART

References considered to be relevant as background to the presently disclosed subject matter are listed below:
Perlman D., Microbiological Conversion of Pregnenolone to Progesterone, 115, p. 529 (1952)

Acknowledgement of the above references herein is not to be inferred as meaning that these are in any way relevant to the patentability of the presently disclosed subject matter.

### BACKGROUND

The gut microbiome is involved in a variety of physiological processes.

Perlman D., Microbiological Conversion of Pregnenolone to Progesterone, 115, p. 529 (1952) describes microbiological Conversion of Pregnenolone to Progesterone.

### GENERAL DESCRIPTION

The present disclosure provides in accordance with some aspects, an effective amount of at least one microorganism for use in a method of modulating 3β-hydroxysteroid dehydrogenase (3β-HSD), wherein the at least one microorganism is a gut microbiome microorganism.

The present disclosure provides in accordance with some other aspects, an effective amount of at least one microorganism for use in a method of modulating production of at least one steroid hormone, wherein said at least one microorganism is a gut microbiome microorganism.

The present disclosure provides in accordance with some further aspects, a pharmaceutical composition comprising a therapeutically effective amount of at least one microorganism, wherein the at least one microorganism is a gut microbiome microorganism, and wherein the composition optionally further comprises at least one pharmaceutically acceptable carrier/s, excipient/s, auxiliaries, and/or diluent/s.

The present disclosure provides in accordance with yet some other aspects, method for treating a disease or condition associated with low steroid hormone levels in a subject in need thereof, the method comprises administering to the subject a therapeutically effective amount of at least one microorganism, wherein the at least one microorganism is a gut microbiome microorganism.

The present disclosure provides in accordance with yet some further aspects, use of an effective amount of at least one microorganism in the preparation of a composition for treating a disorder or condition associated with low steroid hormone levels in a subject in need thereof, wherein the at least one microorganism is a gut microbiome microorganism.

The present disclosure provides in accordance with yet some further aspects, a kit composition comprising at least one microorganism, wherein the at least one microorganism is a gut microbiome microorganism and optionally instructions for treating disorder or condition associated with low steroid hormone levels in a subject in need thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a graph showing fold change of 30-hydroxysteroid dehydrogenase (HSDs) expression level using real-time PCR during growth of Hafnia alvei ATCC 51873 relative to levels of housekeeping genes (HKGs), RecA and transcription termination factor rho (Rho), all expression levels are normalized to RecA levels at the beginning of the assay (time 0). Relative fold-change is calculated from the first sampling point of Hafnia alvei ATCC 51873 fermentation.

### DETAILED DESCRIPTION OF EMBODIMENTS

The gut microbiome refers to a large number of diverse microorganisms within the gastrointestinal tract that are capable of affecting a variety of physiological process, including maintenance of host's health as well as influencing disease development and progression.

The present disclosure is based on a finding that one or more microorganisms from the gut microbiome are capable of expressing an enzyme that is involved in the production of steroid hormone. Specifically, it was found that one or more microorganisms from the gut microbiome express 30-hydroxysteroid dehydrogenase.

As 3β-HSD is involved in biosynthesis of various steroid hormones, including, inter alia, progesterone from pregnenolone, 17α-hydroxyprogesterone from 17α-hydroxypregnenolone, and androstenedione from dehydroepiandrosterone (DHEA), it was suggested by the inventors that one or more microorganisms from the gut microbiome, such as Hafnia alvei ATCC 51873 may be capable of synthesizing at least one steroid hormone. This unique and surprisingly biological pathway that was shown here to occur in microorganism, specifically bacterium from the gut microbiome is highly valuable.

As was shown in Example 1A below, a gut microbiome bacterium, Hafnia alvei ATCC 51873 was shown to be capable of expressing 3β-Hydroxysteroid dehydrogenase (3β-HSD) during culturing. Further as shown in this Example, the amount of the expressed 3β-HSD was higher in the presence of a progesterone precursor, pregnenolone. These data suggested that Hafnia alvei ATCC 51873 is capable of producing progesterone, at least via the activity of 3β-HSD.

Identification of the microorganisms from the gut microbiome and specifically bacteria from the gut microbiome can be done for example by computational tools. To that end, the inventors have used an array of computational tools utilized to process high-throughput sequencing data and obtained high resolution detection and annotation of microbial genes and pathways as well as microbial taxa. This results in the identification of bacteria that uniquely express genes that are involved in modulation of steroid hormones, including, inter alia, expression of 3β-HSD gene that can increase production of at least one steroid hormone, such as progesterone.

It was suggested that a unique and specific microorganisms identified computationally by the inventors, may be administered to a subject, either as each microorganism alone or in specific combinations (denoted herein as microbial consortia), in order to alter the microbiome population and to treat or prevent at least one disorder that is associated with reduced levels of steroid hormone, such as progesterone. It was further suggested that the identified microorganism may be used for diagnostic and prognostic purposes, for example for assessing responsiveness of a subject to treatment and for determining treatment protocols.

Hence, in accordance with some aspects, the present disclosure provides, an effective amount of at least one microorganism for use in a method of modulating 3β-hydroxysteroid dehydrogenase (3β-HSD), wherein said at least one microorganism is a gut microbiome microorganism.

3β-HSD (EC 1.1.1.145) is known as an enzyme that catalyzes the biosynthesis of steroid hormones.

Thus, the present disclosure provides in accordance with some other aspects, an effective amount of at least one microorganism for use in a method of modulating production of at least one steroid hormone, wherein said at least one microorganism is a gut microbiome microorganism.

It should be noted that at times the above features as well as other features of the at least one microorganism described herein below is referred to as the “pathway” and at times the pathway product (direct or indirect) is denoted as “substance”.

It should be also noted that reference to at least one microorganism having one ore more of or at least one of is to be understood as characterization of the at least one microorganism indicating that the at least one microorganism (or one or more) is capable of having and at times having the characteristics (features) as listed.

In the following text, when referring to the at least one microorganism it is to be understood as also referring to the microbial consortium, pharmaceutical compositions, kits, uses and methods disclosed herein. Thus, whenever providing a feature with reference to the at least one microorganism, it is to be understood as defining the same feature with respect to the microbial consortium, the pharmaceutical compositions, kits, uses and methods, mutatis mutandis.

The term microorganism used herein refers in accordance with some embodiments, an isolated microorganism, a purified microorganism, a recombinant microorganism or any combinations thereof.

In some embodiments, the microbial consortium comprises isolated microorganisms. In some embodiments, the microbial consortium comprises purified microorganisms. In some embodiments, the microbial consortium comprises recombinant microorganisms. In some embodiments, the microbial consortium comprises isolated microorganisms, purified microorganisms or any combination thereof.

It should be noted that the recombinant microorganisms of the present invention are microorganisms whose genetic makeup has been altered by deliberate introduction or deletion of genetic elements. The recombinant microorganisms may maintain the functions (cellular processes) of the original microorganism.

In some embodiments, the microorganism is a live microorganism, provided as spores, heat-killed, non-living form of the microorganism, an extract of the organism, a component of the microorganism or any combination thereof.

In some embodiments, the microorganism is a live microorganism. In some other embodiments, the microorganism is provided as spores, heat-killed, non-living form of the microorganism. In some further embodiments, the microorganism is an extract of the microorganism. In yet some further embodiments, the microorganism is a component of the microorganism.

Reference to at least one microorganism and specifically at least one bacterium, also encompasses an isolate, a mutant, spores, enzymes or extracts thereof and/or a conditioned culture medium of the at least one bacterium and/or compounds (components/metabolites) secreted from the at least one bacterium.

When referring to at least one microorganism it should be understood as referring to one microorganism species and/or strain as classified under common scientific classification.

As described herein, the at least one microorganism being the subject of the present disclosure is present in the human microbiome and thus can be obtained by any known method and/or from any source. Accordingly, the at least one microorganism can be isolated and/or purified from any microbiome, such as the human microbiome, by any known method in the art as also detailed below or purified from a biological material (e.g, fecal materials, such as feces or materials isolated from the various segments of the small and large intestines).

The at least one microorganism can be part of a microbial consortium. The term microbial consortium as used herein refers to a mixture/cocktail of microorganisms, including at least one of a bacteria and/or an archaea.

As described herein, the at least one microorganism can be part of a microbial consortium. A microbial consortium is to be understood as comprising two or more microorganisms. When referring to a microbial consortium comprising at least two microorganisms, it should be understood as referring to two different microorganisms (i.e. different strains). The two different microorganisms may be within the same microbial genus or within the microorganism species.

For examples, in case the present disclosure refers to a microbial consortium, the two or more microorganisms can belong both to one genus but to different species within or alternatively to the same genus, the same species but different strains. Alternatively, the two or more microorganisms can belong each to a different genus.

In some embodiments, at least one of the two or more microorganisms is a gut microbiome microorganism being capable of modulating the production of at least one steroid hormone. In some embodiments, at least two of the two or more microorganisms are gut microbiome microorganisms being capable of modulating the production of at least one steroid hormone.

In some embodiments, at least one of the two or more microorganisms is a gut microbiome microorganism being capable of modulating the expression of 3β-HSD. In some embodiments, at least two of the two or more microorganisms are gut microbiome microorganisms being capable of modulating the expression of 3β-HSD.

In some embodiments, the at least one microorganism belongs one of the following genus Hafnia genus, Escherichia genus, Obesumbacterium genus or Sodalis genus.

In some embodiments, the microbial consortium comprises one or more microorganism from the Hafnia genus, Escherichia genus, Obesumbacterium genus, Sodalis genus or combinations thereof.

In some embodiments, the at least one microorganism belongs to Hafnia genus. In some embodiments, the microbial consortium comprises at least one microorganism from the Hafnia genus. Hafnia genus may be denoted by Taxonomy ID: 568. Hafnia is a genus of Gram-negative bacteria.

In some embodiments, the at least one microorganism belongs to Escherichia genus. In some embodiments, the microbial consortium comprises at least one microorganism from the Escherichia genus. Escherichia genus may be denoted by Taxonomy ID: 561.

In some embodiments, the at least one microorganism belongs to Obesumbacterium genus. In some embodiments, the microbial consortium comprises at least one microorganism from the Obesumbacterium genus. Obesumbacterium genus may be denoted by Taxonomy ID: 82982.

In some embodiments, the at least one microorganism belongs to Sodalis genus. In some embodiments, the microbial consortium comprises at least one microorganism from the Sodalis genus. Sodalis genus may be denoted by Taxonomy ID: 1999.

In some embodiments, the at least one microorganism belongs to at least one of the species: Hafnia alvei species, Hafnia paralvei species, Hafnia spp. species, Escherichia coli species, Escherichia spp. species, Obesumbacterium proteus species, Sodalis spp. species or any combinations thereof.

In some embodiments, the at least one microorganism is a microorganism of the Hafnia alvei species. In some embodiments, the microbial consortium comprises at least one microorganism from the Hafnia alvei species. Hafnia alvei species may be denoted by Taxonomy ID: 569.

In some embodiments, the at least one microorganism is a microorganism of the Hafnia paralvei species. In some embodiments, the microbial consortium comprises at least one microorganism from the Hafnia paralvei species. Hafnia paralvei species may be denoted by Taxonomy ID: 546367.

In some embodiments, the at least one microorganism is a microorganism of the Hafnia spp. species. In some embodiments, the microbial consortium comprises at least one microorganism from the Hafnia spp species. Hafnia spp species may be denoted by Taxonomy ID: 1873498.

In some embodiments, the at least one microorganism is a microorganism of the Escherichia coli species. In some embodiments, the microbial consortium comprises at least one microorganism from the Escherichia coli species. Escherichia coli species may be denoted by Taxonomy ID: 562.

In some embodiments, the at least one microorganism is a microorganism of the Escherichia spp. species. In some embodiments, the microbial consortium comprises at least one microorganism from the Escherichia spp. species. Escherichia spp. species may be denoted by Taxonomy ID: 1884818.

In some embodiments, the at least one microorganism is a microorganism of the Obesumbacterium proteus species. In some embodiments, the microbial consortium comprises at least one microorganism from the Obesumbacterium proteus species. Obesumbacterium proteus species may be denoted by Taxonomy ID: 82983.

In some embodiments, the at least one microorganism is a microorganism of the Sodalis spp. species. In some embodiments, the microbial consortium comprises at least one microorganism from the Sodalis spp species. Sodalis spp species may be denoted by Taxonomy ID: 1898979.

In the context of the present disclosure, identification of microorganism from a biological sample of a human subject may be done using any conventional method in the microbiology field. For example, and without being limited to identification of bacteria from a biological sample of a human subject may be done using 16S rRNA (ribosomal RNA) sequencing. Identification of isolated microorganism may be done by conducting similarity analysis between the 16S rRNA gene of the isolated microorganism to a different microorganism's 16S rRNA gene sequences available in a database. This analysis may be done in order to explore the similarity between a given sequence and all of the available sequences in a database and obtaining the best matched sequences by calculation of a score for the examined similarity. Identity analysis may be conducted by any appropriate program, for example Basic Local Alignment Search Tool (BLAST®) using publicly available databases, for example National Center for Biotechnology Information (NCBI).

It should be noted that the GenBank or Refseq accession Nos. provide below either provide the 16S rRNA sequence of the microorganism or the sequence of the entire genome. It should be further noted that a skilled person would know to evaluate the 16S rRNA sequence from the entire genome sequence.

In some embodiments, at least one microorganism comprises 16S rRNA sequences having at least 85%, at least 90%, at least 95%, at least 97%, at least 98%, at least 99% identity with at least one nucleic acid sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21 or SEQ ID NO:22.

In some embodiments, at least one microorganism comprises 16S rRNA sequences having at least 85%, at least 90%, at least 95%, at least 97%, at least 98%, at least 99% identity with at least one nucleic acid sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11.

In some embodiments, at least one microorganism comprises 16S rRNA sequences having at least 85%, at least 90%, at least 95%, at least 97%, at least 98%, at least 99% identity with at least one nucleic acid sequences denoted by SEQ ID NO:2.

In some embodiments, at least one microorganism comprises 16S rRNA sequences having at least 85%, at least 90%, at least 95%, at least 97%, at least 98%, at least 99% identity with at least one nucleic acid sequences denoted by SEQ ID NO:12, SEQ ID NO:13, SEQ ID NO:14, SEQ ID NO:15, SEQ ID NO:16, SEQ ID NO:17 or SEQ ID NO:18.

In some embodiments, at least one microorganism comprises 16S rRNA sequences having at least 85%, at least 90%, at least 95%, at least 97%, at least 98%, at least 99% identity with at least one nucleic acid sequences denoted by SEQ ID NO:19 or SEQ ID NO:20.

In some embodiments, at least one microorganism comprises 16S rRNA sequences having at least 85%, at least 90%, at least 95%, at least 97%, at least 98%, at least 99% identity with at least one nucleic acid sequences denoted by SEQ ID NO:21 or SEQ ID NO:22.

In some embodiments, at least one microorganism comprises 16S rRNA sequences having between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21 or SEQ ID NO:22.

In some embodiments, at least one microorganism comprises 16S rRNA sequences having between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11.

In some embodiments, at least one microorganism comprises 16S rRNA sequences having between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:2.

In some embodiments, at least one microorganism comprises 16S rRNA sequences having between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:12, SEQ ID NO:13, SEQ ID NO:14, SEQ ID NO:15, SEQ ID NO:16, SEQ ID NO:17 or SEQ ID NO:18.

In some embodiments, at least one microorganism comprises 16S rRNA sequences having between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:19 or SEQ ID NO:20.

In some embodiments, at least one microorganism comprises 16S rRNA sequences having between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:21 or SEQ ID NO:22.

The term identity (% identity) as used herein refer to two or more nucleic acid sequences that are the same. In the context of the present disclosure the sequence identity encompasses transcription changes of DNA to RNA, e.g. T and U are considered identical. The identity may exist over a region of a sequence that is considered by those versed in the art as the variable region of the 16S rRNA. In some embodiments, the identity exists over the length the 16S rRNA or a portion thereof of the variable region.

The % identity between two or more nucleic acid sequences is determined for the two or more sequences when compared and aligned for maximum correspondence. In the context of the present disclosure, sequences (nucleic acid) as described herein having % identity are considered to have the same function/activity of the original sequence to which identity is calculated to.

The threshold sequence identity may be 85%, at times 86%, at times 87%, at times 88%, at times 89%, at times 90%, at times 91%, at times 92%, at times 93%, at times 94%, at times 95%, at times 96%, at times 97%, at times 98%, at times 99% with each one of the % identity denoted herein constitute a separate embodiment of the invention.

In some embodiments, the at least one microorganism is Hafnia alvei ATCC 51873. In some embodiments, the microbial consortium comprises at least Hafnia alvei ATCC 51873. Hafnia alvei ATCC 51873 may be denoted by Taxonomy ID: 1002364. The DNA sequence of Hafnia alvei ATCC 51873 is provided by GenBank accession No. AGCI00000000.1. The 16S rRNA sequence of Hafnia alvei ATCC 51873 is denoted as SEQ ID NO:1.

In some embodiments, the at least one microorganism is Hafnia alvei ATCC 13337. In some embodiments, the microbial consortium comprises at least Hafnia alvei ATCC 13337. Hafnia alvei ATCC 13337 may be denoted by Taxonomy ID: 910996. The DNA sequence of Hafnia alvei ATCC 13337 is provided by GenBank accession No. NZ_CP050150.1. The 16S rRNA sequence of Hafnia alvei ATCC 13337 is denoted as SEQ ID NO:2.

In some embodiments, the at least one microorganism is Hafnia alvei FB1. In some embodiments, the microbial consortium comprises at least Hafnia alvei FB1. Hafnia alvei FB1 may be denoted by Taxonomy ID: 1453496. The DNA sequence of Hafnia alvei FB1 is provided by GenBank accession No. NZ_CP009706.1. The 16S rRNA sequence of Hafnia alvei FB1 is denoted as SEQ ID NO:3.

In some embodiments, the at least one microorganism is Hafnia alvei NCTC6578 (also named Hafnia alvei ATCC 9760). In some embodiments, the microbial consortium comprises at least Hafnia alvei NCTC6578. The DNA sequence of Hafnia alvei NCTC6578 is provided by GenBank accession No. NZ_UGHM01000001.1. The 16S rRNA sequence of Hafnia alvei NCTC6578 is denoted as SEQ ID NO:4.

In some embodiments, the at least one microorganism is Hafnia alvei PCM 1202. In some embodiments, the microbial consortium comprises at least Hafnia alvei PCM 1202. The DNA sequence of Hafnia alvei PCM 1202 is provided by GenBank assembly accession No. GCA_004308065.1. The 16S rRNA sequence of Hafnia alvei PCM 1202 is denoted as SEQ ID NO:5.

In some embodiments, the at least one microorganism is Hafnia alvei A23BA. In some embodiments, the microbial consortium comprises at least Hafnia alvei A23BA. The DNA sequence of Hafnia alvei A23BA is provided by GenBank assembly accession No. GCA_011617105.1. The 16S rRNA sequence of Hafnia alvei A23BA is denoted as SEQ ID NO:6.

In some embodiments, the at least one microorganism is Hafnia alvei HUMV-5920. In some embodiments, the microbial consortium comprises at least Hafnia alvei HUMV-5920. The DNA sequence of Hafnia alvei HUMV-5920 is provided by GenBank assembly accession No. GCA_001636255.1. The 16S rRNA sequence of Hafnia alvei HUMV-5920 is denoted as SEQ ID NO:7.

In some embodiments, the at least one microorganism is Hafnia alvei HUMV-FDAARGOS_1038. In some embodiments, the microbial consortium comprises at least Hafnia alvei HUMV-FDAARGOS_1038. The DNA sequence of Hafnia alvei FDAARGOS_1038 is provided by GenBank assembly accession No.

GCA_016403205.1. The 16S rRNA sequence of Hafnia alvei FDAARGOS_1038 is denoted as SEQ ID NO:8.

In some embodiments, the at least one microorganism is Hafnia paralvei ATCC 29927. In some embodiments, the microbial consortium comprises at least Hafnia paralvei ATCC 29927. Hafnia paralvei ATCC 29927 may be denoted by Taxonomy ID: 1354263. The DNA sequence of Hafnia paralvei ATCC 29927 is provided by GenBank accession No. NZ_LXET00000000.1. The 16S rRNA sequence of Hafnia paralvei ATCC 29927 is denoted as SEQ ID NO:9.

In some embodiments, the at least one microorganism is Hafnia paralvei AVS0177. In some embodiments, the microbial consortium comprises at least Hafnia paralvei AVS0177. The DNA sequence of Hafnia paralvei AVS0177 is provided by GenBank accession No. NZ_CP083737.1. The 16S rRNA sequence of Hafnia paralvei AVS0177 is denoted as SEQ ID NO:10.

In some embodiments, the at least one microorganism is Hafnia sp. HMSC23F03. In some embodiments, the microbial consortium comprises at least Hafnia sp. HMSC23F03. Hafnia sp. HMSC23F03 may be denoted by Taxonomy ID: 1581059. The DNA sequence of Hafnia sp. HMSC23F03 is provided by GenBank accession No. NZ_LWPO00000000.1. The 16S rRNA sequence of Hafnia sp. HMSC23F03 is denoted as SEQ ID NO:11.

In some embodiments, the at least one microorganism is Escherichia coli CFT073. In some embodiments, the microbial consortium comprises at least Escherichia coli CFT073. Escherichia coli CFTO73 be denoted by Taxonomy ID: 199310. The DNA sequence of Escherichia coli CFTO73 provided by GenBank accession No. NZ_CP051263.1. The 16S rRNA sequence of Escherichia coli CFTO73 is denoted as SEQ ID NO:12.

In some embodiments, the at least one microorganism is Escherichia coli Nissle 1917. In some embodiments, the microbial consortium comprises at least Escherichia coli Nissle 1917. Escherichia coli Nissle 1917 may be denoted by Taxonomy ID: 316435. The 16S rRNA sequence of Escherichia coli Nissle 1917 is denoted as SEQ ID NO:13.

In some embodiments, the at least one microorganism is Escherichia coli O2:H1 strain HM670. In some embodiments, the microbial consortium comprises at least Escherichia coli O2:H1 strain HM670. Escherichia coli O2:H1 strain HM670 may be denoted by Taxonomy ID: 2079157. The DNA sequence of Escherichia coli O2:H1 strain HM670 provided by GenBank accession No. CP082772.1. The 16S rRNA sequence of Escherichia coli O2:H1 strain HM670 is denoted as SEQ ID NO:14

In some embodiments, the at least one microorganism is Escherichia coli FVEC1302. In some embodiments, the microbial consortium comprises at least Escherichia coli FVEC1302. Escherichia coli FVEC1302 may be denoted by Taxonomy ID: 656379. The DNA sequence of Escherichia coli FVEC1302 provided by GenBank accession No. NZ_ACXH00000000.1. The 16S rRNA sequence of Escherichia coli FVEC1302 is denoted as SEQ ID NO:15.

In some embodiments, the at least one microorganism is Escherichia sp. E4742. In some embodiments, the microbial consortium comprises at least Escherichia sp. E4742. Escherichia sp. E4742 may be denoted by Taxonomy ID: 2044467. The DNA sequence of Escherichia sp. E4742 provided by GenBank accession No. CP040443.1. The 16S rRNA sequence of Escherichia sp. E4742 is denoted as SEQ ID NO:16.

In some embodiments, the at least one microorganism is Escherichia sp. E4694. In some embodiments, the microbial consortium comprises at least Escherichia sp. E4694. Escherichia sp. E4694 may be denoted by Taxonomy ID: 2044464. The 16S rRNA sequence of Escherichia sp. E4694 is denoted as SEQ ID NO:17.

In some embodiments, the at least one microorganism is Escherichia sp. E4702. In some embodiments, the microbial consortium comprises at least Escherichia sp. E4702. Escherichia sp. E4702 may be denoted by Taxonomy ID: 2044465. The 16S rRNA sequence of Escherichia sp. E4702 is denoted as SEQ ID NO:18.

In some embodiments, the at least one microorganism is Obesumbacterium proteus ATCC 12841. In some embodiments, the microbial consortium comprises at least Obesumbacterium proteus ATCC 12841. Obesumbacterium proteus ATCC 12841 may be denoted by Taxonomy ID: 1354268. The DNA sequence of Obesumbacterium proteus ATCC 12841 provided by GenBank accession No. NZ_LXEX00000000.1. The 16S rRNA sequence of Obesumbacterium proteus ATCC 12841 is denoted as SEQ ID NO:19.

In some embodiments, the at least one microorganism is Obesumbacterium proteus PCM 1204. In some embodiments, the microbial consortium comprises at least Obesumbacterium proteus PCM 1204. The DNA sequence of Obesumbacterium proteus PCM 1204 provided by GenBank accession No. NZ_SITH00000000.1. The 16S rRNA sequence of Obesumbacterium proteus PCM 1204 is denoted as SEQ ID NO:20.

In some embodiments, the at least one microorganism is Sodalis sp. dw_96. In some embodiments, the microbial consortium comprises at least Sodalis sp. dw_96. Sodalis sp. dw_96 may be denoted by Taxonomy ID: 2719794. The DNA sequence of Sodalis sp. dw_96 provided by GenBank accession No. NZ_JAAVUT000000000.1. The 16S rRNA sequence of Sodalis sp. dw_96 is denoted as SEQ ID NO:21.

In some embodiments, the at least one microorganism is Sodalis sp. dw_23. In some embodiments, the microbial consortium comprises at least Sodalis sp. dw_23. Sodalis sp. dw_23 may be denoted by Taxonomy ID: 2697027. The DNA sequence of Sodalis sp. dw_23 provided by GenBank accession No. CP075169.1. The 16S rRNA sequence of Sodalis sp. dw_23 is denoted as SEQ ID NO:22.

In some embodiments, the at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03, Escherichia coli CFT073, Escherichia coli Nissle 1917, Escherichia coli O2:H1 strain HM670, Escherichia coli FVEC1302, Escherichia sp. E4742, Escherichia sp. E4694, Escherichia sp. E4702, Obesumbacterium proteus ATCC 12841, Obesumbacterium proteus PCM 1204, Sodalis sp. dw_96, Sodalis sp. dw_23, or any combination thereof.

In some embodiments, the at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03 or any combinations thereof.

In some embodiments, the at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, or any combinations thereof.

In some embodiments, the at least one microorganism is at least one of Hafnia paralvei ATCC 29927, Hafnia paralvei AVS0177 or any combinations thereof.

In some embodiments, the at least one microorganism is Hafnia sp. HMSC23F03.

In some embodiments, the at least one microorganism is at least one of Escherichia coli CFTO73, Escherichia coli Nissle 1917, Escherichia coli O2:H1 strain HM670, Escherichia coli FVEC1302, Escherichia sp. E4742, Escherichia sp. E4694, Escherichia sp. E4702 or any combinations thereof.

In some embodiments, the at least one microorganism is at least one of Escherichia coli CFTO73, Escherichia coli Nissle 1917, Escherichia coli O2:H1 strain HM670, Escherichia coli FVEC1302, or any combinations thereof.

In some embodiments, the at least one microorganism is at least one of Escherichia sp. E4742, Escherichia sp. E4694, Escherichia sp. E4702 or any combinations thereof.

In some embodiments, the at least one microorganism is at least one of Obesumbacterium proteus ATCC 12841, Obesumbacterium proteus PCM 1204 or any combination thereof.

In some embodiments, the at least one microorganism is at least one of Sodalis sp. dw_96, Sodalis sp. dw_23, or any combination thereof.

As detailed above, the at least one microorganism from the microbiome was shown to express and specifically increase expression of 3β-HSD gene. Further and as detailed above, it was suggested that the at least one microorganism can be used in modulating and specifically producing at least one steroid hormone and hence increasing its level (amount, content).

In some embodiments, modulating production of at least one steroid hormone is in view of expression of 3β-HSD. In some embodiments, the at least one steroid hormone is produced by activity of 3β-HSD. In some embodiments, the production of at least one steroid hormone is catalyzed by 3β-HSD.

In some embodiments, the at least one microorganism increase 3β-HSD expression. In some other embodiments, the at least one microorganism increase 3β-HSD expression thereby increase production of at least one steroid hormone.

In some embodiments, the at least one steroid hormone is at least one of a corticosteroids, glucocorticoids, mineralocorticoids, a sex steroid or a combination thereof.

In some embodiments, the at least one steroid hormone is at least one of a corticosteroids.

In some embodiments, the at least one steroid hormone is at least one of a sex steroid.

As used herein a steroid hormone, also known as sex steroid, gonadocorticoid and gonadal steroid, interact with steroid hormone receptors, including, inter alia, androgen, estrogen, and progesterone.

In some embodiments, the at least one steroid hormone is at least one sex steroid.

In some embodiments, the at least one steroid hormone is at least one of progesterone, 16α-hydroxyprogesterone (16α-OHP), 17α-hydroxyprogesterone (17α-OHP), 20α-dihydroprogesterone (20α-DHP), 20β-dihydroprogesterone (20β-DHP), 5α-dihydroprogesterone (5α-DHP), 5β-dihydroprogesterone (5β-DHP), 3β-dihydroprogesterone (3β-DHP), 11-deoxycorticosterone (DOC), and 5α-dihydrodeoxycorticosterone (5α-DHDOC) or any combination thereof.

In some embodiments, the at least one steroid hormone is at least one of progesterone, testosterone, 17α-hydroxyprogesterone and androstenedione.

In some embodiments, the at least one steroid hormone is progesterone, 17α-hydroxyprogesterone or combinations thereof.

In some other embodiments, the at least one steroid hormone is progesterone.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is a gut microbiome microorganism.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03, Escherichia coli CFT073, Escherichia coli Nissle 1917, Escherichia coli O2:H1 strain HM670, Escherichia coli FVEC1302, Escherichia sp. E4742, Escherichia sp. E4694, Escherichia sp. E4702, Obesumbacterium proteus ATCC 12841, Obesumbacterium proteus PCM 1204, Sodalis sp. dw_96, Sodalis sp. dw_23, or any combination thereof.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03 or any combinations thereof.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, or any combinations thereof.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is Hafnia alvei ATCC 51873.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is at least one of Hafnia paralvei ATCC 29927, Hafnia paralvei AVS0177 or any combinations thereof.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is Hafnia sp. HMSC23F03.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is at least one of Escherichia coli CFTO73, Escherichia coli Nissle 1917, Escherichia coli O2:H1 strain HM670, Escherichia coli FVEC1302, Escherichia sp. E4742, Escherichia sp. E4694, Escherichia sp. E4702 or any combinations thereof.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is at least one of Escherichia coli CFTO73, Escherichia coli Nissle 1917, Escherichia coli O2:H1 strain HM670, Escherichia coli FVEC1302, or any combinations thereof.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is at least one of Escherichia sp. E4742, Escherichia sp. E4694, Escherichia sp. E4702 or any combinations thereof.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is at least one of Obesumbacterium proteus ATCC 12841, Obesumbacterium proteus PCM 1204 or any combination thereof.

In accordance with some embodiments that may be considered as aspects of the invention, it is provided an effective amount of at least one microorganism for use in modulating production of progesterone, 17α-hydroxyprogesterone or combinations thereof, wherein the at least one microorganism is at least one of Sodalis sp. dw_96, Sodalis sp. dw_23, or any combination thereof.

As described herein, the at least one microorganism can modulate expression, production and the like.

The term modulation or modulating refers to an effect of altering a biological activity, i.e. a change, modification or the like and encompasses a change that results in activation/increased activity of a as a physiological/cellular process. In some examples, the biological activity is associated with expression 3β-HSD. In some examples, the biological activity is associated with production of at least one steroid hormone, such as, progesterone.

An activator refers to a microorganism that induces, activates, stimulates, increases, facilitates, enhances activation, sensitizes or up regulates at least one physiological/cellular process. Hence, the at least one microorganism may be considered as an activator of a specific process (pathway). Such process can be any known process resulting in production and specifically increased production of at least one steroid hormone, such as progesterone.

As described herein, the present invention encompasses processes which are modulated endogenously in/by the microorganisms and/or processes which are modulated in the host by products being modulated (e.g. produced) in at least one microorganism or in the microbial consortium and secreted or/and modified therefrom to a host. A physiological process as used herein encompasses physical and/or biological and/or chemical events in/by the microorganism and/or in the host.

Processes that are modulated endogenously in the microorganisms, i.e. microbial-endogenous processes are all collectively denoted herein as “microbial-component processes”. The microbial processes may take place in at least one specific type (species or strain) of microorganism (endogenous process).

Processes that take place in the host by the use of a microorganism's components such as a product produced by at least one microorganism in the microbial consortium is denoted herein as “host-component processes”.

It should be noted that as shown herein the at least one microorganism is capable of expressing 3β-HSD gene.

Without being bound by theory, it was suggested that the 33-HSD protein may either be used (at least partially) by at the at least one microorganism in the catalysis of at least one steroid hormone (a process encompassed within “microbial-component processes”). The at least one steroid hormone produced by the at least one microorganism may be secreted to a cell or host.

Without being bound by theory, it was further suggested that the 3β-HSD protein may be secreted (at least partially) to a cell or host (of a subject), and be used in the production/synthesis of at least one steroid hormone (a process encompassed within “host-component processes”).

It should be noted that production of at least one steroid hormone either by the at least one microorganism (microbial-component process), by a cell (e.g. gut host) (host-component process) or any combination of the two may increase the at least one steroid hormone amount and hence the at least one steroid hormone function in the cell in which it is produced, secreted or both. In some embodiments, the at least one microorganism modulate production of progesterone, 17α-hydroxyprogesterone or combinations thereof and hence enhances the levels and functions.

Modulating may occur in vitro (in a cell) or in vivo (in a subject).

Hence, in accordance with some aspects, the present disclosure provides a method for expressing 3β-HSD, the method comprising incubating an effective amount of at least one microorganism in a growth medium, wherein the at least one microorganism is a gut microbiome microorganism.

In some embodiments, the method is an in vitro method.

Hence, it is provided an in vitro method for expressing 3β-HSD, the method comprising incubating an effective amount of at least one microorganism in a growth medium, wherein the at least one microorganism is a gut microbiome microorganism.

As appreciated expression of 3β-HSD by the at least one microorganism may result in 3β-HSD protein.

In some other embodiments, the in vitro method comprising incubating an effective amount of the at least one microorganism under conditions allowing production of at least one steroid hormone, wherein the at least one microorganism is a gut microbiome microorganism.

In some embodiments, the in vitro method comprising incubating an effective amount of the at least one microorganism under conditions allowing expression of 30-HSD gene.

In some other embodiments, the in vitro method comprising incubating an effective amount of the at least one microorganism under conditions allowing production of 33-HSD protein.

In some embodiments, the in vitro method comprising incubating an effective amount of the at least one microorganism under conditions allowing production of at least one steroid hormone.

The conditions can be selected and adopted by a person skilled in the art.

Such conditions include, for example, incubation time, incubation temperature, type of growth medium.

In some embodiments, the growth medium is Brain Heart Infusion broth (BHI), de Man, Rogosa & Sharpe media (MRS), Fluid Thioglycollate Medium (FTM).

In some embodiments, the growth medium comprises a precursor of at least one steroid hormone. In some embodiments, the growth medium comprises pregnenolone.

In some embodiments, the at least one microorganism is grown for at least about 2 hours, at times at least about 4 hours, at times at least about 8 hours, at times at least about 12 hours, at times at least about 16 hours, at times at least about 20 hours, at times at least about 24 hours, at times at least about 30 hours, at times at least about 36 hours, at times at least about 40 hours, at times at least about 42 hours, at times at least about 46 hours, at times at least about 48 hours. In some embodiments, the at least one microorganism is grown for about 48 hours.

In some embodiments, the incubation temperature may be between about 25° C. and 37° C.

In some examples, the conditions for expressing 33-HSD gene include growing the at least one microorganism as described in Example 1 below.

In some embodiments, the method comprises obtaining the medium after the incubation. As appreciated, the medium after the incubation comprises the at least one microorganism and at least one of the 3β-HSD gene, 3β-HSD protein or the 3β-HSD gene and protein.

In accordance with some embodiments, the in vitro method comprising incubating an effective amount of the at least one microorganism under conditions allowing production of at least one steroid hormone, wherein the at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03, Escherichia coli CFT073, Escherichia coli Nissle 1917, Escherichia coli O2:H1 strain HM670, Escherichia coli FVEC1302, Escherichia sp. E4742, Escherichia sp. E4694, Escherichia sp. E4702, Obesumbacterium proteus ATCC 12841, Obesumbacterium proteus PCM 1204, Sodalis sp. dw_96, Sodalis sp. dw_23, or any combination thereof.

In accordance with some embodiments, the in vitro method comprising incubating an effective amount of the at least one microorganism under conditions allowing production of at least one steroid hormone, wherein the at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03 or any combinations thereof.

In accordance with some embodiments, the in vitro method comprising incubating an effective amount of the at least one microorganism under conditions allowing production of at least one steroid hormone, wherein the at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, or any combinations thereof.

In accordance with some embodiments, the in vitro method comprising incubating an effective amount of the at least one microorganism under conditions allowing production of at least one steroid hormone, wherein the at least one microorganism is Hafnia alvei ATCC 51873.

In accordance with some embodiments, the in vitro method comprising incubating an effective amount of the at least one microorganism under conditions allowing production of at least one steroid hormone, wherein the at least one microorganism is at least one of Hafnia paralvei ATCC 29927, Hafnia paralvei AVS0177 or any combinations thereof.

In accordance with some embodiments, the in vitro method comprising incubating an effective amount of the at least one microorganism under conditions allowing production of at least one steroid hormone, wherein the at least one microorganism is Hafnia sp. HMSC23F03.

In accordance with some embodiments, the in vitro method comprising incubating an effective amount of the at least one microorganism under conditions allowing production of at least one steroid hormone, wherein the at least one microorganism is at least one of Escherichia coli CFTO73, Escherichia coli Nissle 1917, Escherichia coli O2:H1 strain HM670, Escherichia coli FVEC1302, Escherichia sp. E4742, Escherichia sp. E4694, Escherichia sp. E4702 or any combinations thereof.

In accordance with some embodiments, the in vitro method comprising incubating an effective amount of the at least one microorganism under conditions allowing production of at least one steroid hormone, wherein the at least one microorganism is at least one of Escherichia coli CFTO73, Escherichia coli Nissle 1917, Escherichia coli O2:H1 strain HM670, Escherichia coli FVEC1302, or any combinations thereof.

In accordance with some embodiments, the in vitro method comprising incubating an effective amount of the at least one microorganism under conditions allowing production of at least one steroid hormone, wherein the at least one microorganism is at least one of Escherichia sp. E4742, Escherichia sp. E4694, Escherichia sp. E4702 or any combinations thereof.

In accordance with some embodiments, the in vitro method comprising incubating an effective amount of the at least one microorganism under conditions allowing production of at least one steroid hormone, wherein the at least one microorganism is at least one of Obesumbacterium proteus ATCC 12841, Obesumbacterium proteus PCM 1204 or any combination thereof.

In accordance with some embodiments, the in vitro method comprising incubating an effective amount of the at least one microorganism under conditions allowing production of at least one steroid hormone, wherein the at least one microorganism is at least one of Sodalis sp. dw_96, Sodalis sp. dw_23, or any combination thereof.

In accordance with some embodiments, the in vitro method can be used for production of at least one of progesterone, 16α-hydroxyprogesterone (16α-OHP), 17α-hydroxyprogesterone (17α-OHP), 20α-dihydroprogesterone (20α-DHP), 200-dihydroprogesterone (20β-DHP), 5α-dihydroprogesterone (5α-DHP), 50-dihydroprogesterone (5β-DHP), 3β-dihydroprogesterone (3β-DHP), 11-deoxycorticosterone (DOC), and 5α-dihydrodeoxycorticosterone (5(α-DHDOC) or any combination thereof.

In accordance with some embodiments, the in vitro method can be used for production of at least one of progesterone, testosterone, 17α-hydroxyprogesterone and androstenedione.

In accordance with some embodiments, the in vitro method can be used for production of at least one of progesterone, 17α-hydroxyprogesterone or combinations thereof.

In accordance with some embodiments, the in vitro method can be used for production of progesterone.

In some embodiments, the method comprises obtaining the medium after the incubation. As appreciated, the medium after the incubation comprises the at least one microorganism and at least one (i) of the 3β-HSD gene, (ii) 33-HSD protein, (iii) at least one steroid hormone or (iv) any combination thereof.

The medium after the incubation according to the invention can be in accordance with some embodiments treated to isolate at least one (i) of the 33-HSD gene, (ii) 30-HSD protein, (iii) at least one steroid hormone or (iv) any combination thereof.

The medium after the incubation according to the invention can be in accordance with some embodiments treated to isolate at least one steroid hormone.

The medium after the incubation according to the invention can be in accordance with some embodiments treated to isolate at least one of progesterone, testosterone, 17α-hydroxyprogesterone and androstenedione.

The medium after the incubation according to the invention can be in accordance with some embodiments treated to isolate at least one of progesterone, 17α-hydroxyprogesterone or combinations thereof.

The medium after the incubation according to the invention can be in accordance with some embodiments treated to isolate progesterone.

Hence, in some aspects, it is provided a 3β-HSD protein obtained by a process comprising incubating an effective amount of the at least one microorganism under conditions allowing production of 3β-HSD protein, wherein the at least one microorganism is a gut microbiome microorganism.

In some other aspects, it is provided a 33-HSD protein obtainable by a process comprising incubating an effective amount of the at least one microorganism under conditions allowing production of 3β-HSD protein, wherein the at least one microorganism is a gut microbiome microorganism.

The 3β-HSD protein may be obtained or obtainable together with the at least one microorganism.

In some embodiments, the process comprising incubating at least one microorganism in a growth medium as noted above.

In some aspects, it is provided at least one steroid hormone obtained by a process comprising incubating an effective amount of the at least one microorganism under conditions allowing production of at least one steroid hormone, wherein the at least one microorganism is a gut microbiome microorganism.

Hence, in some aspects, it is provided at least one steroid hormone obtainable by a process comprising incubating an effective amount of the at least one microorganism under conditions allowing production of at least one steroid hormone as described herein wherein the at least one microorganism is a gut microbiome microorganism.

The at least one steroid hormone may be obtained or obtainable together with the at least one microorganism.

In some embodiments, the process comprising incubating at least one microorganism in a growth medium as noted above.

In some embodiments, the process comprising incubating at least one microorganism in a growth medium for at least one microorganism is grown for at least about 2 hours, at times at least about 4 hours, at times at least about 8 hours, at times at least about 12 hours, at times at least about 16 hours, at times at least about 20 hours, at times at least about 24 hours, at times at least about 30 hours, at times at least about 36 hours, at times at least about 40 hours, at times at least about 42 hours, at times at least about 46 hours, at times at least about 48 hours.

In some embodiments, the process comprising incubating at least one microorganism in a growth medium for about 48 hours.

In some embodiments, the process comprising incubating at least one microorganism in a growth medium comprising pregnenolone.

As described herein, increased production of at least one steroid hormone, including, inter alia, progesterone by the at least one microorganism of the invention may improve clinical outcomes when the at least one microorganism is administrated to a subject in need thereof.

Hence, it is provided in accordance with some aspects, at least one microorganism for use to increase the amount of at least one steroid hormone in a subject in need thereof, wherein the at least one microorganism is a gut microbiome microorganism. In accordance with some embodiments, the subject is suffering from a disorder or condition associated with decreased level of at least one steroid hormone.

As appreciated, increasing the amount of at least one steroid hormone is via production of the at least one steroid hormone by the at least one microorganism.

Further and in accordance with some other aspects, it is provided a method for increasing the amount of at least one steroid hormone in a subject in need thereof, wherein the method comprising administrating an effective amount of at least one microorganism, wherein the at least one microorganism is a gut microbiome microorganism. In accordance with some embodiments, the subject is suffering from a disorder or condition associated with decreased level of at least one steroid hormone.

The disorder or condition associated with decreased level of at least one steroid hormone may any hormonal disorder.

In accordance with some other aspects, it is provided at least one microorganism for use in treating a disorder or condition associated with decreased level of at least one steroid hormone in a subject in need thereof, wherein said at least one microorganism is a gut microbiome microorganism.

In accordance with some further aspects, it is provided a method for treating, disorder or condition associated with decreased level of at least one steroid hormone in a subject in need thereof. More specifically, the method may comprise administering to the subject a therapeutically effective amount of at least one microorganism as described herein.

The disorder or condition associated with decreased level of at least one steroid hormone may required to increase steroid hormone levels in a subject in need thereof.

In some embodiments, the disorder or condition associated with decreased level of at least one steroid hormone is at least one of irregular menstrual cycles, absent menstrual cycles, mood changes, hot flashes, night sweats, vaginal dryness, headaches, migraines, repeated miscarriages, intestinal permeability associated syndromes, abdominal pain, endometriosis, bacterial vaginosis, functional gastrointestinal disorder, infertility, visceral pain, visceral hypersensitivity (also known as visceral hyperalgesia) or any combination thereof.

In some embodiments, the method is for treating irregular menstrual cycles.

In some embodiments, the method is for treating absent menstrual cycles.

In some embodiments, the method is for treating mood changes.

In some embodiments, the method is for treating hot flashes.

In some embodiments, the method is for treating night sweats.

In some embodiments, the method is for treating vaginal dryness.

In some embodiments, the method is for treating headaches.

In some embodiments, the method is for treating migraines.

In some embodiments, the method is for treating miscarriage.

In some embodiments, the method is for treating intestinal permeability associated syndromes.

In some embodiments, the method is for treating abdominal pain.

In some embodiments, the method is for treating endometriosis.

In some embodiments, the method is for treating bacterial vaginosis.

In some embodiments, the method is for treating functional gastrointestinal disorder.

In some embodiments, the at least one microorganism used by the methods of the invention comprises at least one microorganism that belongs to at least one of the species: Hafnia alvei species, Hafnia paralvei species, Hafnia spp. species, Escherichia coli species, Escherichia spp. species, Obesumbacterium proteus species, Sodalis spp. species or any combinations thereof.

In some embodiments, the at least one microorganism used by the methods of the invention is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03, Escherichia coli CFTO73, Escherichia coli Nissle 1917, Escherichia coli O2:H1 strain HM670, Escherichia coli FVEC1302, Escherichia sp. E4742, Escherichia sp. E4694, Escherichia sp. E4702, Obesumbacterium proteus ATCC 12841, Obesumbacterium proteus PCM 1204, Sodalis sp. dw_96, Sodalis sp. dw_23, or any combination thereof.

In some embodiments, the at least one microorganism used by the methods of the invention is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, or any combinations thereof.

In some embodiments, the at least one microorganism used by the methods of the invention is Hafnia alvei ATCC 51873.

In some embodiments, the at least one microorganism used by the methods of the invention is at least one of Hafnia paralvei ATCC 29927, Hafnia paralvei AVS0177 or any combinations thereof.

In some embodiments, the at least one microorganism used by the methods of the invention is Hafnia sp. HMSC23F03.

In some embodiments, the at least one microorganism used by the methods of the invention is at least one of Escherichia coli CFTO73, Escherichia coli Nissle 1917, Escherichia coli O2:H1 strain HM670, Escherichia coli FVEC1302, or any combinations thereof.

In some embodiments, the at least one microorganism used by the methods of the invention is at least one of Escherichia sp. E4742, Escherichia sp. E4694, Escherichia sp. E4702 or any combinations thereof.

In some embodiments, the at least one microorganism used by the methods of the invention is at least one of Obesumbacterium proteus ATCC 12841, Obesumbacterium proteus PCM 1204 or any combination thereof.

In some embodiments, the at least one microorganism used by the methods of the invention is at least one of Sodalis sp. dw_96, Sodalis sp. dw_23, or any combination thereof.

In some embodiments, the methods of the invention comprises administration of at least one microorganism that belongs to at least one of the species: Hafnia alvei species, Hafnia paralvei species, Hafnia spp. species, Escherichia coli species, Escherichia spp. species, Obesumbacterium proteus species, Sodalis spp. species or any combinations thereof to a subject in need thereof for treating a disorder or condition associated with low steroid hormone levels.

In some embodiments, the methods of the invention comprises administration of at least one microorganism that belongs to at least one of the species: Hafnia alvei species, Hafnia paralvei species, Hafnia spp. species, Escherichia coli species, Escherichia spp. species, Obesumbacterium proteus species, Sodalis spp. species or any combinations thereof to a subject in need thereof for treating a disorder or condition associated with low progesterone levels.

In some embodiments, the methods of the invention comprises administration of at least one microorganism that belongs to at least one of the species: Hafnia alvei species, Hafnia paralvei species, Hafnia spp. species, Escherichia coli species, Escherichia spp. species, Obesumbacterium proteus species, Sodalis spp. species or any combinations thereof to a subject in need thereof for treating at least one of irregular menstrual cycles, absent menstrual cycles, mood changes, hot flashes, night sweats, vaginal dryness, headaches, migraines, repeated miscarriages, intestinal permeability associated syndromes, abdominal pain, endometriosis, bacterial vaginosis, functional gastrointestinal disorder, infertility, visceral pain or any combination thereof.

In some embodiments, the methods of the invention comprises administration of at least one microorganism that belongs to at least one of the species: Hafnia alvei species, to a subject in need thereof for treating a disorder or condition associated reduced levels of at least one steroid hormone.

In some embodiments, the methods of the invention comprises administration of at least one microorganism that belongs to at least one of the species: Hafnia alvei species, to a subject in need thereof for treating a disorder or condition associated reduced levels of progesterone.

In some embodiments, the methods of the invention comprises administration of at least one microorganism that belongs to at least one of the species: Hafnia alvei species, to a subject in need thereof for treating a disorder or condition associated with low steroid hormone levels. In some embodiments, the at least one steroid hormone production is catalyzed by 3β-HSD. In some embodiments, the at least one steroid hormone is progesterone.

In some embodiments, the methods of the invention comprises administration of at least one microorganism that belongs to Hafnia alvei species, to a subject in need thereof for treating at least one of irregular menstrual cycles, absent menstrual cycles, mood changes, hot flashes, night sweats, vaginal dryness, headaches, migraines, repeated miscarriages, intestinal permeability associated syndromes, abdominal pain, endometriosis, bacterial vaginosis, functional gastrointestinal disorder, infertility, visceral pain or any combination thereof.

In some embodiments, the methods of the invention comprises administration of at least one microorganism being at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, or any combination thereof to a subject in need thereof for treating a disorder or condition associated with low levels of at least one steroid hormone. In some embodiments, the at least one steroid hormone production is catalyzed by 3β-HSD. In some embodiments, the at least one steroid hormone is progesterone.

In some embodiments, the methods of the invention comprises administration of at least one microorganism being at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, or any combination thereof to a subject in need thereof for treating at least one of irregular menstrual cycles, absent menstrual cycles, mood changes, hot flashes, night sweats, vaginal dryness, headaches, migraines, repeated miscarriages, intestinal permeability associated syndromes, abdominal pain, endometriosis, bacterial vaginosis, functional gastrointestinal disorder, infertility, visceral pain or any combination thereof.

In some embodiments, the methods of the invention comprises administration of at least Hafnia alvei ATCC 51873 to a subject in need thereof for treating a disorder or condition associated with low steroid hormone levels. In some embodiments, the at least one steroid hormone production is catalyzed by 3β-HSD. In some embodiments, the at least one steroid hormone is progesterone.

In some embodiments, the methods of the invention comprises administration of at least Hafnia alvei ATCC 51873 to a subject in need thereof for treating at least one of irregular menstrual cycles, absent menstrual cycles, mood changes, hot flashes, night sweats, vaginal dryness, headaches, migraines, repeated miscarriages, intestinal permeability associated syndromes, abdominal pain, endometriosis, bacterial vaginosis, functional gastrointestinal disorder, infertility, visceral pain or any combination thereof.

As used herein irregular menstrual cycles refers to short or long cycles that may affect the menstrual cycle. The average menstrual cycle lasts 28 days and hence short or long cycle are typically defined as menstrual cycle of shorter than 21 days or longer than 35 days.

As used herein absent menstrual cycles refers to lack of 3 menstrual cycles in a row.

As used herein mood changes encompasses depression, anxiety, irritability and insomnia which are typically worsen before the cycle.

As used herein, a functional gastrointestinal disorder refers to any disorder or pathologically condition which affect different parts of the gastrointestinal tract and involve visceral hypersensitivity and motility disturbances.

The functional gastrointestinal disorder is considered in accordance with some embodiments, as having combination of the following: motility disturbance, visceral hypersensitivity, altered mucosal and immune function, altered gut microbiota, altered ENS processing and altered central nervous system (CNS) processing. It is of note that the functional gastrointestinal disorder encompasses related symptoms such as pain and bloating.

In some embodiments, the functional gastrointestinal disorder is a functional dyspepsia (FD). Functional dyspepsia is associated with one or more symptoms of bloating, burning, pain, or early fullness or prolonged fullness in the upper digestive tract, nausea, vomiting, burping, weight loss, sour taste in the mouth, that are present for at least one month.

In some embodiments, the functional dyspepsia is at least one of postprandial distress syndrome (PDS) or epigastric pain syndrome (EPS).

In some embodiments, the functional gastrointestinal disorder is a centrally mediated disorders of gastrointestinal pain.

In some embodiments, the centrally mediated disorders of gastrointestinal pain is centrally mediated abdominal pain syndrome (CAPS), narcotic bowel syndrome (NBS)/opioid-induced GI hyperalgesia or combination thereof.

Centrally mediated abdominal pain syndrome (CAPS), is a condition of abdominal (belly) pain that is long-term or repeated pain (keeps coming back), which is not related to changes in bowel pattern—constipation and/or diarrhea.

Narcotic bowel syndrome (NBS) is a condition that is characterized by worsening abdominal pain in the context of escalating or continuous opioid therapy.

In some embodiments, the functional gastrointestinal disorder is a bowel disorder.

In some embodiments, the bowel disorder is at least one of irritable bowel syndrome (TBS), functional constipation, functional diarrhea, functional abdominal bloating/distension, unspecified functional bowel disorder, opioid-induced constipation or combination thereof.

In some embodiments, the functional gastrointestinal disorder is IBS.

IBS is characterized by a variety of symptoms including abdominal pain and changes in the frequency and consistency of bowel movements.

In some embodiments, the IBS is at least one of IBS with predominant constipation (IBS-C), IBS with predominant diarrhea (IBS-D), IBS with mixed bowel habits (IBS-M), IBS unclassified (IBS-U) or combination thereof.

There are number of in vivo models that have been developed for studying the different conditions of the IBS-related GI dysfunctions. Amongst these models, different kinds of stressors, for instance, psychological and physical stimuli appear to play critical roles in the development of IBS and the maintenance of the disorder. Additionally, models that implement feces transplantation of IBS patients into germ-free animals can also stimulate the developments of IBS phenotypes such as visceral hypersensitivity and abnormal bowel movement—for more details “Pathogenesis, Experimental Models and Contemporary Pharmacotherapy of Irritable Bowel Syndrome: Story About the Brain-Gut Axis” S.W. Tsang et al, the context of which is incorporated by reference.

In some embodiments, the disorder or condition associated with decreased level of at least one steroid hormone is not a functional gastrointestinal disorder.

The methods of the invention comprise in accordance with some embodiments, administration of the at least one microorganism of the invention with an additional treatment.

The at least one microorganism and the additional treatment or treatments may be administrated simultaneously or sequentially.

As appreciated, the additional treatment is selected, for example, to be compatible with the at least one microorganism for the purpose of improving the effect of the at least one microorganism. In some embodiments, the additional treatment is a state-of-the-art treatment in any condition. For example, nonsteroidal anti-inflammatory drugs (NSAIDs) ibuprofen (Advil, Motrin IB, others), Gonadotropin-releasing hormone (Gn-RH) agonists and antagonists, oral contraceptives, Aromatase inhibitors, Selective serotonin reuptake inhibitors (SSRIs), Serotonin-norepinephrine reuptake inhibitors (SNRIs), Tricyclic antidepressants, Monoamine oxidase inhibitors (MAOIs), Atypical antidepressants (bupropion, mirtazapine, nefazodone, trazodone and vortioxetine).

The at least one microorganism may be administrated to a human subject by any method known in the art and described herein. In accordance with some embodiments, the methods of the invention comprise administration the at least one microorganism or a composition comprising the same by oral administration. In accordance with some embodiments, the methods of the invention comprise administration the at least one microorganism or a composition comprising the same that is formulated for delivery to the intestine. The at least one microorganism or a composition may be formulated for example into a capsule, granules, sachets, tablet, suppositories, food or beverage or any of the like.

As described herein, the present disclosure relates to an effective amount of at least one microorganism. In aspects wherein the present disclosure relates to more than one microorganism, i.e. at least two microorganisms, such two microorganisms are referred herein as microbial consortium. In some embodiments, the microbial consortium comprises the same or equivalent amounts of the microorganisms forming the consortium.

In some further embodiments, the microbial consortium comprises different amounts of the microorganisms forming the consortium.

In some embodiments, the total number per one dose (cell count/amount/colony forming units-CFUs/Optical density measurement) of each microorganism either alone or forming the microbial consortium is at least 1×10², at least 1×10³, at least 10⁴, at least 1×10⁵, at times at least 1×10⁶, at times at least 1×10⁷, at times at least 1×10⁸.

In some embodiments, the total number per one dose (cell count/amount/colony forming units-CFUs/Optical density measurement) of each microorganism either alone or forming the microbial consortium between about 1×10⁸ and about 5×101° C. FU, between about 2×10⁸ and about 4×10¹⁰, between about 3×10⁸ and about 3×10¹⁰, between about 5×10⁸ and about 1×10¹⁰.

In some embodiments, the microbial consortium comprises the same or equivalent amounts of the microorganisms forming the consortium.

In some further embodiments, the microbial consortium comprises different amounts of the microorganisms forming the consortium. In some embodiments, the total number (cell count/amount/colony forming units-CFUs) of each one of microorganisms forming the microbial consortium is between about 1×10³ to about 1×10¹², between 1×10⁵ to about 1×10¹⁰, 1×10⁸ to about 5×10¹⁰, between 2×10⁸ to about 4×10¹⁰, between 3×10⁸ to about 3×10¹⁰, between 5×10⁸ to about 1×10¹⁰.

In some embodiments, the total number (cell count/amount/colony forming units-CFUs) of microorganisms forming the microbial consortium is between about 1×10³ to about 1×10¹², between 1×10⁵ to about 1×10¹⁰, 1×10⁸ to about 5×10¹⁰, between 2×10⁸ to about 4×10¹⁰, between 3×10⁸ to about 3×10¹⁰, between 5×10⁸ to about 1×10¹⁰.

As detailed above, the at least one microorganism can be identified in, purified or isolated from the microbiome of a reference subject, for example by collecting a biological sample. The biological sample may be any sample from which the population of microorganisms can be isolated, for example, feces. In yet another embodiment, the sample may be a biopsy of human organs or tissue, specifically, a gut biopsy.

The at least one microorganism and/or the microbial consortium may be formulated in a variety of forms, depending on the storage, administration etc. Non-limiting forms include solid, dry form, for example, in a lyophilized powder, gel form, a suspension, a cell lysate or extract. In some embodiments, at least one microorganism and/or the microbial consortium may be suspended in a liquid medium (such as PBS or saline) and used in a suspension form.

The at least one microorganism and/or the microbial consortium of the invention may be used in the preparation of a pharmaceutical formulation/compositions/suspension or in the manufacture of a formulation/compositions/suspension.

As such, formulation/compositions/suspension of the invention may comprise apart from a therapeutically effective amount of at least one microorganism and/or a microbial consortium of the invention, at least one additional component as detailed herein.

In a further aspect, the invention relates to a composition (suspension or formulation) comprising at least one microorganism or a microbial consortium of the invention for uses and methods as described herein.

As described herein, the at least one microorganism, the microbial consortium and/or the suspension/composition comprising the same may form a kit of the invention. In general, the composition and/or the suspension and/or kit described herein comprising the at least one microorganism or the microbial consortium form part of this invention. It should be noted that the forms described herein for the at least one microorganism and/or the microbial consortium per se apply for the composition and/or the suspension and/or kit comprising the microbial consortium.

In yet some further embodiments, the composition of the invention may optionally further comprise at least one of pharmaceutically acceptable carrier/s, excipient/s, additive/s diluent/s and adjuvant/s. As used herein “pharmaceutically acceptable carrier” includes any and all solvents, dispersion media, coatings and the like.

Aqueous suspensions may further contain substances which increase the viscosity of the suspension including, for example, sodium carboxymethylcellulose, sorbitol and/or dextran. The suspension may also contain stabilizers.

It should be understood that in addition to the ingredients particularly mentioned above, the formulations may also include other agents conventional in the art having regard to the type of formulation in question.

The at least one microorganism, the at least one microorganism, the microbial consortium, and/or any suspensions/compositions comprising the same can be administered and dosed by the methods of the invention as described below, in accordance with medical procedures known in the art. For example, the suspensions/compositions used in the methods and kits of the invention, described herein below, may be adapted for administration by various modes of administration that are known in the art including, for example, systemic, parenteral, intraperitoneal, transdermal, oral (including buccal or sublingual), rectal, topical (including buccal or sublingual), vaginal, intranasal and any other appropriate routes.

Specific examples include but not limited to, injection (e.g., using a subcutaneous, intramuscular, intravenous, or intradermal injection), intranasal administration, vaginal and oral administration.

In some embodiments, the at least one microorganism of the invention and/or any suspensions/compositions comprising the same may be formulated for oral administration.

In some embodiments, the at least one microorganism of the invention and/or any suspensions/compositions comprising the same may be formulated for delivery to the intestine. In some embodiments, the at least one microorganism of the invention and/or any suspensions/compositions comprising the same may be formulated into food or a beverage.

In some embodiments, the at least one microorganism of the invention and/or any suspensions/compositions comprising the same may be formulated to be contained within a carrier.

In some embodiments, the at least one microorganism of the invention and/or any suspensions/compositions comprising the same is enterically coated.

In some embodiments, the methods of the invention may comprise administrating the at least one microorganism, the microbial consortium of the invention, compositions or kits comprising the same and optionally an additional treatment, as a single one-time dose, as a single daily dose or multiple daily doses, preferably, every 1 to 7 days. It is specifically contemplated that such application may be carried out once or several times in the lifetime of a patient, once, twice, thrice, four times, five times or six times daily, or may be performed once daily, once every 2 days, once every 3 days, once every 4 days, once every 5 days, once every 6 days, once every week, two weeks, three weeks, four weeks or even more than a month.

The invention further provides the use of at least one microorganism in the preparation of a composition for treating, preventing, inhibiting, reducing or delaying the onset of disease or condition associated with low steroid hormone levels in a subject in need thereof, wherein the at least one microorganism is a gut microbiome microorganism.

In another aspect, the invention provides a kit comprising at least one microorganism of the invention in the preparation of a composition for treating a disease or condition associated with low steroid hormone levels, in a subject in need thereof.

In some embodiments, the kits described herein may include a composition/suspension as described, as an already prepared dosage form ready for administration or, alternatively, can include the at least one microorganism or a composition as described as a solid pharmaceutical composition (e.g. in a lyophilized form) that can be reconstituted with a solvent to provide a liquid dosage form. The kit of the invention may additionally comprise instructions for using the kit in treating a disease or condition associated with low steroid hormone levels.

The methods of treatment provided herein involve administration of the at least one microorganism of the invention in a therapeutically effective amount. The term “effective amount” as used herein is that determined by such considerations as are known to the man of skill in the art. The amount must be sufficient to improve the disorder or conditions described herein. Dosing is dependent on the severity of the symptoms and on the responsiveness of the subject to at least one microorganism of the invention. Medically professionals can easily determine the optimum dosage, dosing methodology and repetition rates, taking into consideration the age, sex, weight and state of the disease of the subject to be treated, will determine the dose.

As use herein “therapeutically effective amount” means an amount of at least one microorganism, a microbial consortium and/or a composition comprising the same which provides a medical benefit as noted by the clinician or other qualified observer.

The present invention provides methods for treating a disease, disorder or condition associated with low steroid hormone levels. The term “treatment or prevention” refers to the complete range of therapeutically positive effects of administrating to a subject including inhibition, reduction of, alleviation of, and relief from, a disease associated with low steroid hormone levels, or undesired side effects of such.

As used herein, “disease”, “disorder”, “condition” and the like, as they relate to a subject's health, are used interchangeably and have meanings ascribed to each and all of such terms.

The present invention relates to methods for the treatment of subjects, or patients, in need thereof. By “patient” or “subject in need” it is meant any organism who may be affected by the above-mentioned conditions, and to whom the treatment methods herein described are desired. It should be further noted that particularly in case of human subject, administering of the compositions of the invention to the patient includes both self-administration and administration to the patient by another person.

In some embodiments, the subject is a female subject. In some embodiments, the subject is a female subject suffering from a hormonal disorder or condition. In some embodiments, the subject is a female subject. In some embodiments, the subject is a female subject suffering from a hormonal disorder or condition associated with low steroid hormone levels.

The invention provides methods for treating a disease associated with low steroid hormone levels, and further relates to disorders associated or related to a such disorder. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology.

It should be noted that indicate “increasing” or “enhancing” for example the expression or the levels of 3β-HSD transcripts, it is meant that such increase or enhancement may be an increase or elevation of between about 10% to 100% of the expression and/or stability of 3β-HSD. The terms “increase”, “augmentation” and “enhancement” as used herein relate to the act of becoming progressively greater in size, amount, number, or intensity. Particularly, an increase of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the expression and/or level (amount, content) of at least one steroid hormone as compared to a suitable control. It should be further noted that increase or elevation may be also an increase of about 2 to 10⁶ folds or more. Still further, it should be appreciated that the increase of the levels or expression of said 3β-HSD may be either in translation or the stability of said 3β-HSD. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. Therefore, the term increase refers to an increase of about 2, 3, 4, 5, 6, 7, 8, 9, 10, 20, 30, 40, 50, 60, 70, 80, 90, 100, 200, 300, 400, 500, 600, 700, 800, 900, 1000 folds or more.

It should be noted that a suitable control refers at times to the respective pathway/substance without the at least one microorganism described herein (being the subject of the present invention).

“Expression”, as used herein generally refers to the process by which gene-encoded information is converted into functional gene product that enables the synthesis of structures present and operating in the cell. Therefore, according to the invention “expression” of a gene, specifically, may refer to transcription into a polynucleotide, and following downstream processes such as translation into a protein, or even posttranslational modification of the protein. Protein stability, as used herein, refers to the physical (thermodynamic) stability, and chemical stability of the protein and relates to the net balance of forces, which determine whether a protein will be in its native folded conformation or a denatured state.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this invention may be presented in a range format. The description of a range should be considered to have specifically disclosed all the possible sub ranges as well as individual numerical values within that range. For example, description of a range such as from 1 to 6 or between 1 and 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

It should be noted that the term “at least one” refers to one or more of the following and encompasses any one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more of the specified information. For example, when referring to at least one microorganism, it should be understood to refer to one or more microorganism and to encompasses any one of 1, 2, 3, 4, 5, or more microorganisms.

It is to be understood that the terminology used herein is used for the purpose of describing particular embodiments only and not intended to be limiting since the scope of the present invention will be limited only by the appended claims and equivalents thereof.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It should be noted that the various embodiments and examples detailed herein in connection with various aspects of the invention may be applicable to one or more aspects disclosed herein. It should be further noted that any embodiment described herein, for example, related to components of the microbial consortium, may be applied separately or in various combinations. Various embodiments and aspects of the present invention as delineated hereinabove and as claimed in the claims section below find experimental support in the following examples. The phrases “in another embodiment” or any reference made to embodiment as used herein do not necessarily refer to different embodiment, although it may. Thus, various embodiments of the invention can be combined (from the same or from different aspects) without departing from the scope of the invention.

TABLE 1
List of Sequences

| SEQ ID | |
|---|---|
| NO: | Details |
| 1 | Gene encoding 16S rRNA of Hafnia alvei ATCC 51873 |
| 2 | Gene encoding 16S rRNA of Hafnia alvei ATCC 13337 |
| 3 | Gene encoding 16S rRNA of Hafnia alvei FB1 |
| 4 | Gene encoding 16S rRNA of Hafnia alvei NCTC6578 |
| 5 | Gene encoding 16S rRNA of Hafnia alvei PCM |
| 6 | Gene encoding 16S rRNA of Hafnia alvei A23BA |
| 7 | Gene encoding 16S rRNA of Hafnia alvei HUMV-5920 |
| 8 | Gene encoding 16S rRNA of Hafnia alvei HUMV-FDAARGOS_1038 |
| 9 | Gene encoding 16S rRNA of Hafnia paralvei ATCC 29927 |
| 10 | Gene encoding 16S rRNA of Hafnia paralvei AVS0177 |
| 11 | Gene encoding 16S rRNA of Hafnia sp. HMSC23F03 |
| 12 | Gene encoding 16S rRNA of Escherichia coli CFT073 |
| 13 | Gene encoding 16S rRNA of Escherichia coli Nissle 1917 |
| 14 | Gene encoding 16S rRNA of Escherichia coli O2:H1 strain HM670 |
| 15 | Gene encoding 16S rRNA of Escherichia coli FVEC1302 |
| 16 | Gene encoding 16S rRNA of Escherichia sp. E4742 |
| 17 | Gene encoding 16S rRNA of Escherichia sp. E4694 |
| 18 | Gene encoding 16S rRNA of Escherichia sp. E4702 |
| 19 | Gene encoding 16S rRNA of Obesumbacterium proteus ATCC 12841 |
| 20 | Gene encoding 16S rRNA of Obesumbacterium proteus PCM 1204 |
| 21 | Gene encoding 16S rRNA of Sodalis sp. dw_96 |
| 22 | Gene encoding 16S rRNA of Sodalis sp. dw_23 |

### NON-LIMITING EXAMPLES

### Example 1: In Vitro Progesterone Synthesis

### Example 1A: Progesterone Synthesis by Hafnia alvei ATCC 51873

In these experiments, the ability of Hafnia alvei ATCC 51873 to synthesize progesterone was tested by means of quantifying the expression of the progesterone producing gene 30-hydroxysteroid dehydrogenase (HSDs).

Hafnia alvei ATCC 51873 cells were grown with and without the progesterone precursor, pregnenolone, over a course of 48 hours. Cells were harvested, lysed and RNA was extracted. Lysis and purification of RNA were performed according to RNA protect protocol for Enzymatic Lysis, and Proteinase K Digestion of bacteria followed by RNA purification using RNeasy Mini Kit (Qiagen, catalog no. 74104). As specified in this kit, Proteinase K from Tritirachium (Sigma, catalog no. P2308) was used for peptide digestion, and enzymatic lysis was carried out using Lysozyme from chicken egg white (Sigma, catalog no. L6876). Purification was completed with removal of excess DNA using RNase-Free Dnase Set (Qiagen, catalog no. 79254). Pure RNA was stored in −20° C. until cDNA library preparation. cDNA library was constructed using HighCapacity cDNA Reverse Transcription Kit (Thermo Fisher Scientific, catalog no. 4368814). Real-time PCR was performed on StepOnePlus™ Real-Time PCR System (Applied Biosystems, 4376600), using PowerUp™ Syber Green™ mastermix (Applied Biosystems, A25742). RecA and Rho were used as housekeeping genes (HKGs) controls. Results were presented as fold change, calculated according to the acceptable method of 2\hat{ }(−ΔΔ Ct). ΔCt was calculated by first subtracting the average Ct value of the gene of interest from the housekeeping gene, RecA. ΔΔ Ct was then calculated by subtracting the Δ Ct value of the treated sample from the control samples that did not contain the precursor of progesterone, pregnenolone.

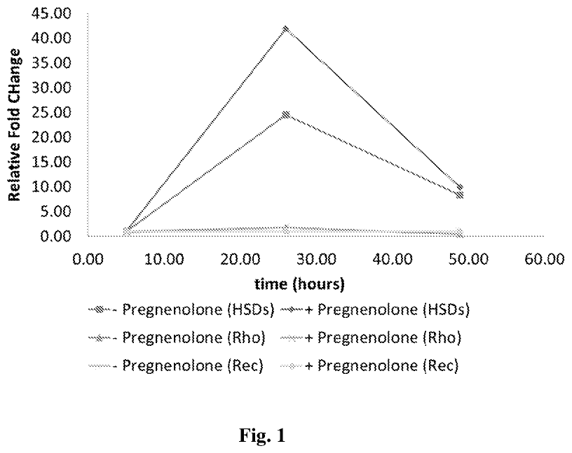

The results presented in FIG. 1 demonstrate that the enzyme transcripts of HSDs were increased along incubation time and reached a maximum after 24 hours of incubation. At that time, enzyme transcripts of HSDs were approximately two folds higher in samples originating from Hafnia alvei ATCC 51873 fermentation with pregnenolone as compared to Hafnia alvei ATCC 51873 fermentation without pregnenolone. These results show that Hafnia alvei ATCC 51873 is capable of synthesizing progesterone.

### Example 1B: Progesterone Identification

The ability of Hafnia alvei strains and others to synthesize progesterone is tested by means of quantifying of progesterone levels using bioanalytical LC-MS/MS.

Hafnia alvei ATCC 51873 cells is grown with and without the progesterone precursor, pregnenolone, at 37° C. for up to 48 hours. During the 48 hours of fermentation, samples are taken for progesterone quantification. Cells are separated by centrifugation (10 min, 3220 g, 4° C.), and the cultured media is filtration through syringe filters (13 mm diameter, 0.22 μm pore size). Filtered media and cells are stored frozen at −80° C. until LC-MS/MS analysis.

### Example 2: In Vivo Visceral Pain/Visceral Hypersensitivity Models

### Example 2A: Trinitrobenzene Sulfonic Acid (TNBS) Model

Male Sprague-Dawley rats are used in this study. Under anesthesia (ketamine, 80 mg/kg i.p.; acepromazine, 12 mg/kg i.p.), the injections of TNBS (50 mg/kg; 1.5 ml/kg) or saline (1.5 ml/kg) is performed into the proximal colon (1 cm from the cecum). After surgery, animals are individually housed in polypropylene cages and kept in a regulated environment. TNBS-induced rectocolitis change the threshold to colonic distension (CRD), demonstrating a visceral hypersensitivity.

### Example 2B: The Maternal Separation (MS) Model

The stress consists in removing the rat pups from their mother for 3 h per day during the first two weeks of life. Since maternal care affects the hypothalamic-pituitary-adrenal (HPA) axis and the cognitive and emotional functions, the MS causes stable changes in the central nervous system of these animals. At the level of the large intestine, MS promotes, in the adult animals, the development of a condition characterized by visceral hypersensitivity to colorectal distension (CRD) and colonic mast cell hyperplasia often concentrates close to nerve endings that are two typical signs of IBS. Additionally, colonic motor function in response to stress is also enhanced in these animals.

### Example 2C: In Vivo Vaginosis Model

Gardnerella vaginalis is one of the most frequently isolated bacterial species in bacterial vaginosis. Mice are anaesthetized with isofluorane and inoculated vaginally with 5×10⁷ CFU G. vaginalis in 20 mL sterile PBS. Vaginal washes are collected by flushing vaginas with sterile PBS. G. vaginalis titers is determined from washes by preparing 10-fold serial dilutions in PBS (in the anaerobic chamber) and spotting each dilution in quadruplicate onto 1 mg/mL streptomycin selection plates.

1. A composition comprising an effective amount of at least one gut microbiome microorganism and a means for increasing 3β-hydroxysteroid dehydrogenase (3β-HSD) expression.

2. The composition according to claim 1, wherein said at least one microorganism produces at least one steroid hormone.

3. (canceled)

4. The composition according to claim 2, wherein said at least one steroid hormone is at least one of progesterone, 16α-hydroxyprogesterone (16α-OHP), 17α-hydroxyprogesterone (17α-OHP), 20α-dihydroprogesterone (20α-DHP), 20β-dihydroprogesterone (20β-DHP), 5α-dihydroprogesterone (5α-DHP), 5β-dihydroprogesterone (5β-DHP), 3β-dihydroprogesterone (3β-DHP), 11-deoxycorticosterone (DOC), and 5α-dihydrodeoxycorticosterone (5α-DHDOC) or any combination thereof.

5. The composition according to claim 4, wherein said at least one steroid hormone is progesterone.

6. The composition according to claim 1, wherein said at least one microorganism belongs to at least one of the species: Hafnia alvei species, Hafnia paralvei species, Hafnia spp. species, Escherichia coli species, Escherichia spp. species, Obesumbacterium proteus species, Sodalis spp. species or any combinations thereof.

7-13. (canceled)

14. The composition according to claim 1, wherein said at least one microorganism is characterized by having a sequence identity of at least 85% with at least one sequence denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21 or SEQ ID NO:22.

15. The composition according to claim 1, wherein said at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03, Escherichia coli CFT073, Escherichia coli Nissle 1917, Escherichia coli O2:H1 strain HM670, Escherichia coli FVEC1302, Escherichia sp. E4742, Escherichia sp. E4694, Escherichia sp. E4702, Obesumbacterium proteus ATCC 12841, Obesumbacterium proteus PCM 1204, Sodalis sp. dw_96, Sodalis sp. dw23, or any combination thereof.

16. The composition according to claim 1, wherein said at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, or any combinations thereof, optionally said at least one microorganism is at least Hafnia alvei ATCC 13337.

17-25. (canceled)

26. A method for treating, preventing, inhibiting, reducing or delaying the onset of a disease or condition associated with low steroid hormone levels in a subject in need thereof, said method comprises administering to said subject a therapeutically effective amount of at least one microorganism, wherein said at least one microorganism is a gut microbiome microorganism.

27. The method according to claim 26, wherein said at least one steroid hormone is at least one of progesterone, 16α-hydroxyprogesterone (16α-OHP), 17α-hydroxyprogesterone (17α-OHP), 20α-dihydroprogesterone (20α-DHP), 20β-dihydroprogesterone (20β-DHP), 5α-dihydroprogesterone (5α-DHP), 5β-dihydroprogesterone (50β-DHP), 3β-dihydroprogesterone (3β-DHP), 11-deoxycorticosterone (DOC), and 5α-dihydrodeoxycorticosterone (5α-DHDOC) or any combination thereof.

28. The method according to claim 27, wherein said at least one steroid hormone is progesterone.

29. The method according to claim 26, wherein said disease or condition is at least one of at least one of irregular menstrual cycles, absent menstrual cycles, mood changes, hot flashes, night sweats, vaginal dryness, headaches, migraines, repeated miscarriages, intestinal permeability associated syndromes, abdominal pain, endometriosis, bacterial vaginosis, functional gastrointestinal disorder, infertility, visceral pain or any combination thereof.

30. The method according to claim 26, wherein said at least one microorganism belongs to at least one of the species: Hafnia alvei species, Hafnia paralvei species, Hafnia spp. species, Escherichia coli species, Escherichia spp. species, Obesumbacterium proteus species, or any combinations thereof.

31. The method according to claim 26, wherein said at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03, Escherichia coli CFT073, Escherichia coli Nissle 1917, Escherichia coli O2:H1 strain HM670, Escherichia coli FVEC1302, Escherichia sp. E4742, Escherichia sp. E4694, Escherichia sp. E4702, Obesumbacterium proteus ATCC 12841, Obesumbacterium proteus PCM 1204, Sodalis sp. dw_96, Sodalis sp. dw23, or any combination thereof, optionally wherein said at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS 1038, or any combinations thereof, optionally wherein said at least one microorganism is at least Hafnia alvei ATCC 13337.

32-45. (canceled)

46. A method for treating, preventing, inhibiting, reducing or delaying the onset of a disease or condition in a subject in need thereof, said method comprises administering to said subject a therapeutically effective amount of at least one gut microbiome microorganism, wherein said at least one microorganism produces at least one steroid hormone.

47. The method according to claim 46, wherein said at least one steroid hormone is at least one of progesterone, 16α-hydroxyprogesterone (16α-OHP), 17α-hydroxyprogesterone (17α-OHP), 20α-dihydroprogesterone (20α-DHP), 20β-dihydroprogesterone (20β-DHP), 5α-dihydroprogesterone (5α-DHP), 5β-dihydroprogesterone (5β-DHP), 3β-dihydroprogesterone (3β-DHP), 11-deoxycorticosterone (DOC), and 5α-dihydrodeoxycorticosterone (5α-DHDOC) or any combination thereof.

48. The method according to claim 47, wherein said at least one steroid hormone is progesterone.

49. The method according to claim 46, wherein said disease or condition associated with low steroid hormone levels, optionally wherein said disease or condition is at least one of at least one of irregular menstrual cycles, absent menstrual cycles, mood changes, hot flashes, night sweats, vaginal dryness, headaches, migraines, repeated miscarriages, intestinal permeability associated syndromes, abdominal pain, endometriosis, bacterial vaginosis, functional gastrointestinal disorder, infertility, visceral pain or any combination thereof.

50. The method according to claim 46, wherein said at least one microorganism belongs to at least one of the species: Hafnia alvei species, Hafnia paralvei species, Hafnia spp. species, Escherichia coli species, Escherichia spp. species, Obesumbacterium proteus species, or any combinations thereof.

51. The method according to claim 46, wherein said at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, Hafnia paralvei ATCC 29927, Hafnia paralvei AVS0177, Hafnia sp. HMSC23F03, Escherichia coli CFT073, Escherichia coli Nissle 1917, Escherichia coli O2:H1 strain HM670, Escherichia coli FVEC1302, Escherichia sp. E4742, Escherichia sp. E4694, Escherichia sp. E4702, Obesumbacterium proteus ATCC 12841, Obesumbacterium proteus PCM 1204, Sodalis sp. dw_96, Sodalis sp. dw_23, or any combination thereof, optionally wherein said at least one microorganism is at least one of Hafnia alvei ATCC 13337, Hafnia alvei ATCC 51873, Hafnia alvei FB1, Hafnia alvei NCTC6578, Hafnia alvei PCM 1202, Hafnia alvei A23BA, Hafnia alvei HUMV-5920, Hafnia alvei FDAARGOS_1038, or any combinations thereof, optionally wherein said at least one microorganism is at least Hafnia alvei ATCC 13337.
